(19) United States
(12) Patent Application Publication
(10) Pub. No.: US 2007/0043389 A1
(43) Pub. Date: Feb. 22, 2007

(54) System for treating chronic total occlusion caused by lower extremity arterial disease

(75) Inventor: Larry Earl Shindelman, Princeton, NJ (US)

Correspondence Address: MCCARTER & ENGLISH, LLP, FOUR GATEWAY CENTER, 100 MULBERRY STREET, NEWARK, NJ, 07102, US

(73) Assignee: ShinTech, LLC

(21) Appl. No.: 11/197,968

(22) Filed: Aug. 5, 2005

(51) Int. Cl. A61M 29/00

(52) U.S. Cl. 606/194

(57) ABSTRACT

A system for the treatment of lower extremity arterial chronic total occlusion (CTO) incorporates remote access of the guide-wire, at least one specifically shaped catheter, and a wire-capture dilation balloon catheter. A capture balloon catheter serves to capture the wire used to traverse the CTO. The capture balloon has a lumen with two axial openings and a radial opening. The capture balloon enters the vascular body from a first opening along a first guide-wire until the balloon is adjacent the CTO. A second guide-wire is advanced from a second opening in the vascular body that is located on an opposite side of the CTO. After the first guide-wire is removed, the second guide-wire is advanced through the funnel-shaped opening in the balloon, then through the radial opening of the lumen and through the lumen so as to advance out of the first opening of the vascular body. A conventional treatment balloon can then be advanced on the second guide-wire to the CTO for treatment.

### FIELD OF THE INVENTION

The present invention relates to dilation type balloon catheters, and diagnostic catheters for use in the treatment of stenotic regions within the arterial circulation. More particularly, the present invention relates to systems and methods for the treatment of chronic total occlusion (CTO) of the arterial circulation occurring in the lower extremities.

### BACKGROUND OF THE INVENTION

The arterial circulation is a system of tubes, comprised of a wall that defines a channel or lumen therein through which blood flows. In Peripheral Arterial Disease (PAD), the arterial wall becomes thickened and results in a corresponding reduction in the available area of the lumen through which blood flows. This reduction in the arterial lumen is called a stenosis. In the lower extremities the thickening of the arterial wall is typically diffuse in nature, and can progress from a stenosis to a blockage or CTO of the arterial lumen. In addition to affecting the arteries of the lower extremities, PAD can affect all the arteries of the arterial system, leading to an increase risk of gangrene, heart attack, stroke and kidney disease.

One way to treat an arterial stenosis is with the use of a dilation balloon catheter, so as to widen the available area of the lumen through which blood flows. A guide-wire is placed percutaneously (through the skin), from a remote puncture site, into the lumen of the arterial system. Under X-ray control this guide-wire is negotiated through the arterial system, through areas of arterial thickening, and through the area of critical stenosis. The dilation balloon is tracked over this guide-wire to the area of critical arterial stenosis, whereupon inflation of the balloon with pressurized fluid, presses the inner area of arterial narrowing toward the outer wall of the blood vessel. The narrowed lumen now enlarges to the manufactured size of the balloon. The balloon dilation catheter is deflated and removed, leaving the available area of the arterial lumen enlarged to allow for the passage of an increased volume of blood.

The opportunity to treat lower extremity PAD is limited by the ability to gain successful guide-wire access through the area of arterial disease. In the treatment of a focal stenosis, guide-wire access is typically straightforward. In diffuse and complex arterial stenosis, however, guide-wire access is more difficult, and most problematic with chronic total occlusions (CTO).

In particular, in the case of CTO, the physician will insert a guide-wire into the arterial lumen, then pass that wire through the arterial lumen to the area of arterial disease. At the point of CTO, the physician will attempt to push the guide-wire through the occlusion by passing the wire from the arterial lumen proximal (upstream) to the occlusion, through the occlusion, and then returning the guide-wire to the arterial lumen distal (downstream) to the area of occlusion. In cases of CTO, when the guide-wire reaches the point of occlusion, it typically does not pass through the center of the occlusion, but “dissects” into the thickened arterial wall just proximal to the CTO. In this dissection plane, with the aid of a catheter, the guide-wire can traverse the area of the CTO. Once the guide-wire is distal to the area of CTO, while remaining within the dissection plane (within the thickened arterial wall) the physician attempts to return the leading edge of the guide-wire to the arterial lumen. With the leading edge of the guide-wire returned to the arterial lumen (distal to the CTO), the dilation balloon catheter is tracked over the wire, and positioned at the area of blockage. Once in place, the dilation balloon is inflated. Pushing outward against the occlusion, recanalization of the artery is established by the dilation balloon, with a luminal connection between the proximal arterial portion and the distal portion of the artery.

In the known systems, once the guide-wire traverses the CTO in the dissection plane, there is great difficulty and complexity involved in returning the guide-wire to the arterial lumen distal to the CTO. This difficulty often leads to failure to gain distal arterial luminal position of the wire, resulting in failure to successfully recanalize the area of CTO, leaving open surgical revascularization as the only alternative treatment option.

### SUMMARY OF THE INVENTION

The shortcomings and disadvantages of the prior art discussed above are overcome by providing an improved catheter system for positioning a guide wire through a treatment site within a vascular body. More particularly, the catheter system includes a first catheter having carrier, which includes a lumen extending therethrough, and an inflatable balloon, which is attached to the carrier so as to be carried thereby. The balloon is expandable from a deflated position to an inflated position in response to the introduction of pressurized fluid into the balloon. The balloon is also provided with an opening formed in an exterior surface of the balloon. The opening permits communication between the exterior surface and the lumen. In accordance with the present invention, the catheter system also includes a second catheter having a portion adjacent an end thereof. The portion of the second catheter is sized and shaped so as to be positioned adjacent the opening of the balloon when the balloon is in its inflated position.

In use, the first catheter is advanced to a treatment site through a vascular body from an upstream side of the treatment site. The second catheter is also advanced to the treatment site through the vascular body from a downstream side of the treatment site. The first catheter is engaged with the second catheter within the vascular body adjacent the treatment site by inflating the balloon. A guide wire is then fed from the second catheter into the first catheter. Thereafter, the first and second catheters are removed from the vascular body, thereby leaving the guide wire extending through the treatment site. The guide wire is used to advance a treatment balloon to the treatment site for treating a CTO condition existing therein.

### BRIEF DESCRIPTION OF THE DRAWINGS

For a more complete understanding of the present invention, reference is made to the following detailed description of an exemplary embodiment, considered in conjunction with the accompanying drawings, in which:

FIG. 1 is a perspective schematic illustration of a system for facilitating proper axial positioning of a capture balloon and associated guide-wires to facilitate treatment of a CTO within vascular bodies in accordance with an exemplary embodiment of the present invention, the system including a balloon assembly, an angled catheter, and plural guide-wires;

FIG. 2 is a side cross-sectional view of the balloon assembly and the angled catheter of the system illustrated in FIG. 1;

FIG. 3 is a side elevational view of the balloon assembly of FIGS. 1 and 2 that shows certain radio-opaque markings used for alignment purposes;

FIG. 4 is a longitudinal cross-sectional view of an occluded region of a vessel showing the system of FIG. 1, except that the balloon assembly is uninflated and the angled catheter has been replaced by a straight catheter;

FIG. 5 is a cross-sectional view similar to that of FIG. 4, except that the straight catheter has been replaced by the angled catheter of FIGS. 1 and 2;

FIG. 6 is a schematic representation of how the apparatus of FIG. 5 would appear to a practitioner utilizing a radioscope display to confirm proper orientation and positioning of the angled catheter and the uninflated balloon assembly relative to each other;

FIG. 7 is a cross-sectional view similar to that of FIG. 5, except that the balloon assembly has now been inflated, causing the complete docking of the angled catheter and the balloon assembly;

FIG. 8 is a schematic representation of how the apparatus of FIG. 7 would appear to a practitioner utilizing a radioscope display to confirm proper coupling of the angled catheter and the now-inflated balloon assembly;

FIG. 9 is an enlarged-scale cross-sectional view of the completely docked angled catheter and balloon assembly of FIG. 7, a guide-wire being shown within the catheter;

FIG. 10 is a cross-sectional view similar to FIG. 9, except that the guide-wire has been advanced through the angled catheter and into the balloon assembly;

FIG. 11 is a cross-sectional view similar to FIG. 10, except that the angled catheter has not been completely docked with the balloon assembly; and

FIG. 12 is a cross-sectional view similar to FIG. 5, showing the balloon assembly in a deflated state and the captured guide-wire advancing further upstream through the balloon assembly.

### DETAILED DESCRIPTION OF THE INVENTION

For the purposes of the discussion below, “proximal” is defined as closer to the heart. Conversely, “distal” is defined as further from the heart. Additionally, the “downstream” direction in an artery is defined as the ordinary direction of blood flow (i.e., away from the heart) within the artery, whereas the “upstream” direction in an artery is defined as being opposite the “downstream” direction therein (i.e., toward the heart).

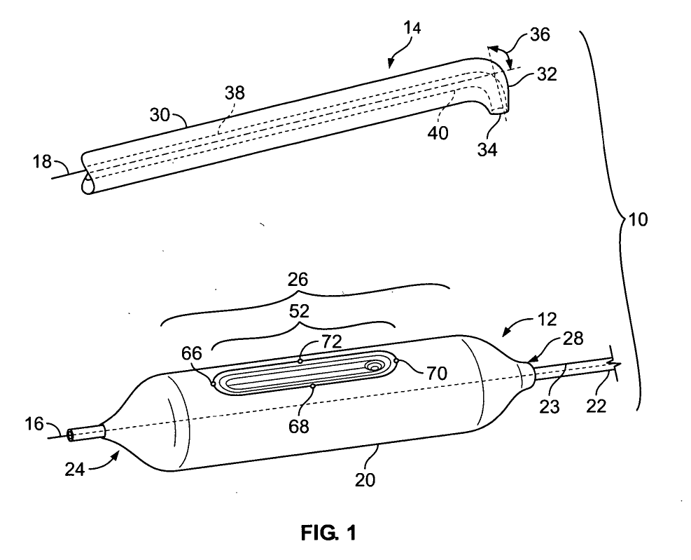

FIG. 1 is a perspective view of a system 10 for treating patients suffering from chronic total occlusion (hereinafter “CTO”) occurring in the lower extremities, in accordance with a first embodiment of the present invention. The system 10, which may be used in conjunction with the inventive methods described hereinbelow, includes a balloon assembly 12, an angled catheter 14, and first and second guide-wires 16, 18, respectively, both of which are of conventional construction. For purposes of clarity, the angled catheter 14 is shown in a scale somewhat larger than that of the balloon assembly 12.

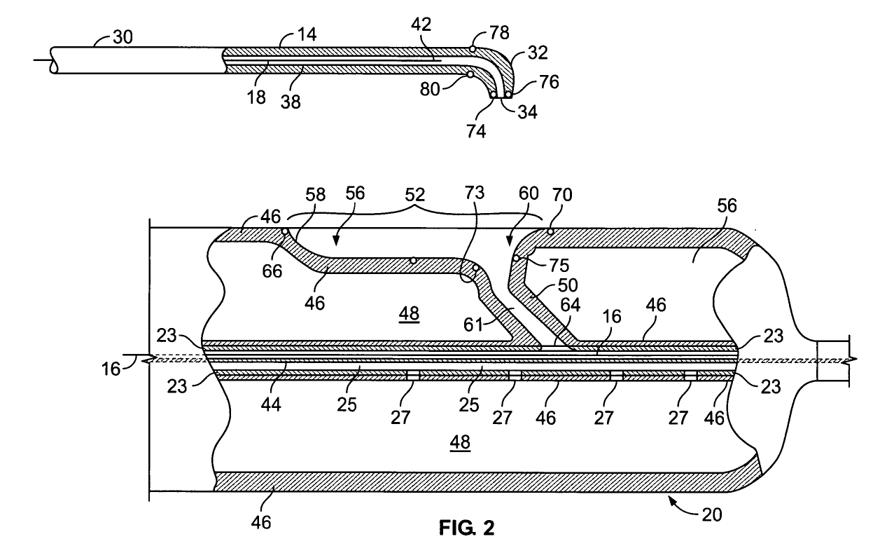

The balloon assembly 12 includes a balloon 20 (shown in a cigar-shaped inflated state), and an elongate tubular body 22 (i.e., a carrier). The balloon 20, which may also be referred to herein as a “capture balloon”, has a first end 24, a generally cylindrical middle portion 26, and a second end 28, and is attached to the elongate body 22 at both the first end 24 and the second end 28. The elongate body 22 is a flexible structure of conventional construction that is used to deliver/retrieve the balloon 20, and to permit the balloon 20 to be remotely inflated and deflated. For such purposes, the elongate body 22 is equipped with an axial lumen 23 (see FIG. 2) sized to accommodate the first guide wire 16, and a wall 44 used to create a separate internally-disposed passage 25 that is hydraulically coupled to the balloon 20 so as to permit a conventional inflation fluid to be delivered to and/or drained from the balloon 20 via holes 27 which are formed therein.

The angled catheter 14 (see FIG. 1) is of a construction similar in many respects to that of a straight catheter, but with some differences. For example, the angled catheter 14 includes an elongate portion 30 and a tapered end portion 32 (the latter terminating at a tip 34 of relatively small diameter), but the tapered end portion 32 is disposed at an angle 36 to the elongate portion 30, rather than being axially aligned therewith. Also, the tapered end portion 32 of the angled catheter 14 is conical at the tip 34, rather than rounded. Further, the angled catheter 14 includes a lumen 38 (see FIG. 1) which is sized to accommodate the second guide-wire 18. More particularly, the lumen 38 extends through the elongate portion 30 and the tapered end portion 32 and terminates at an opening which is formed in the tip 34 and which faces downwardly.

Referring now to FIGS. 1 and 2, the balloon 20 includes certain structures and other features enabling a competent practitioner to cause the balloon 20 to receive the tapered end portion 32 of the angled catheter 14 within a vascular body (e.g., a blood vessel), and to further receive or “capture” an end 42 of the second guide-wire 18. The balloon assembly 12 is further configured, particularly when used in a manner and for purposes to be described more fully hereinafter, to guide the end 42 of the second guide-wire 18 in a smooth and convenient fashion through the balloon 20, and into and through the lumen 23 of the elongate body 22. In this regard, the balloon 20 includes exterior walls 46, which can be considered generally to define an inflatable interior region 48 of the balloon 20. The balloon 20 also includes channel walls 50, as well as a trough 52 which opens up to the exterior surface of the balloon 20 for receiving the tapered end portion 32 of the angled catheter 14. More particularly, the trough 52, which is defined by the exterior walls 46 and/or the channel walls 50 of the balloon 20, is formed in the balloon middle portion 26 along a border or outer perimeter of the balloon 20.

The trough 52 of the balloon 20 features a capture zone 56 adjacent to the outer perimeter of the balloon 20, which includes a scalloped region 58. The scalloped region 58 is formed from the exterior walls 46 of the balloon 20 and is generally concave, relatively shallow, and elongated axially. The scalloped region 58 needs a depth that is preferably at least as deep as the length of the tip 34 of the angle catheter 14 (which is typically about 2 mm, but may be varied according to need). A funnel-shaped opening 60 is also formed from the channel walls 50 and extends inwardly in a generally radial direction from the trough 52 to the elongate body 20. More particularly, the funnel-shaped opening 60 includes a channel 61 (see FIG. 2) which is in a slanted orientation.

Still referring to FIG. 2, the axial lumen 23, which extends through the elongate body 22, is sized to accommodate the first guide-wire 16. As can be seen in FIG. 2, the funnel-shaped opening 60 is oriented relative to the axial lumen 23 at an angle less than 90° so as to facilitate passage of a guide-wire from the funnel-shaped opening 60 into the axial lumen 23. In this regard, the funnel-shaped opening 60 communicates with the axial lumen 23 through an aperture 64 formed in an tubular wall of the elongate body 22.

With reference to FIGS. 1 and 2, the balloon 20 and the angled catheter 14 are each equipped with small, discrete portions of radio-opaque material that are embedded at selected locations in the structural material of each such component. More particularly, the balloon 20 includes small radio-opaque portions in the form of markers 66, 68, 70, 72, which are arranged in spaced relation around the outer perimeter of the trough 52, and markers 73, 75, which are arranged around an entry section of the funnel-shaped opening 60. Also, the angled catheter 14 includes small, discrete radio-opaque portions in the form of markers 74, 76 disposed on opposite longitudinal sides of the tip 34, and markers 78, 80 disposed on opposite axial sides of the elongate portion 30 adjacent the angle 32. The significance of the number and arrangement of these radio-opaque markers will be described in detail hereinafter.

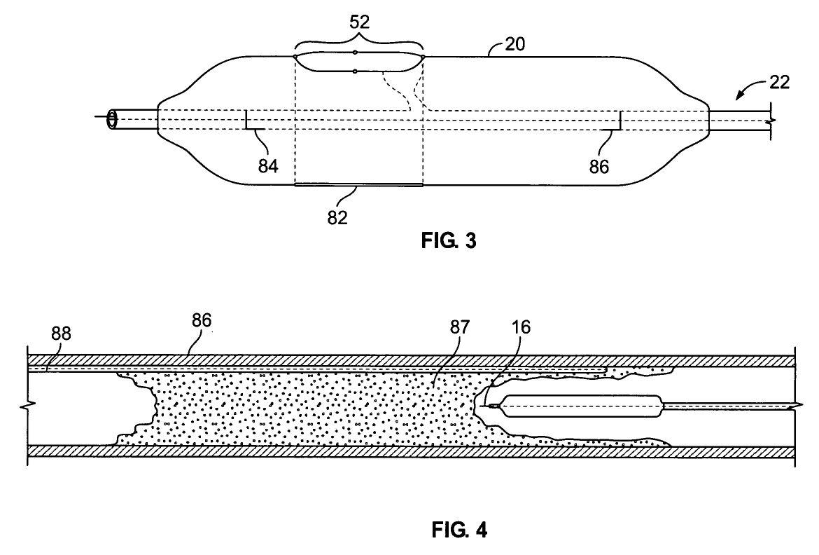

FIG. 3 shows that a lower portion 82 of the balloon 20 is coated and/or constructed of a radio-opaque material. The elongate body 22 also has a plurality of radio-opaque markers 84, 86, each of which has an L-shape and each of which is positioned on a side surface of the elongate body 22 to facilitate alignment of the trough 52 with the tip 34 of the angled catheter 14, as will be explained in greater detail hereinbelow.

As described below with reference to FIGS. 4 to 12, in operation, a competent practitioner can use the system 10 of FIGS. 1 to 3 to improve the axial positioning of the second guide-wire 18 within a totally occluded region (i.e., a treatment site) of a blood vessel. As described above, good axial positioning of a guide-wire improves the chances that a later-placed treatment balloon (not shown) will, when inflated, compress the blockage against the vessel wall in approximately equal amounts.

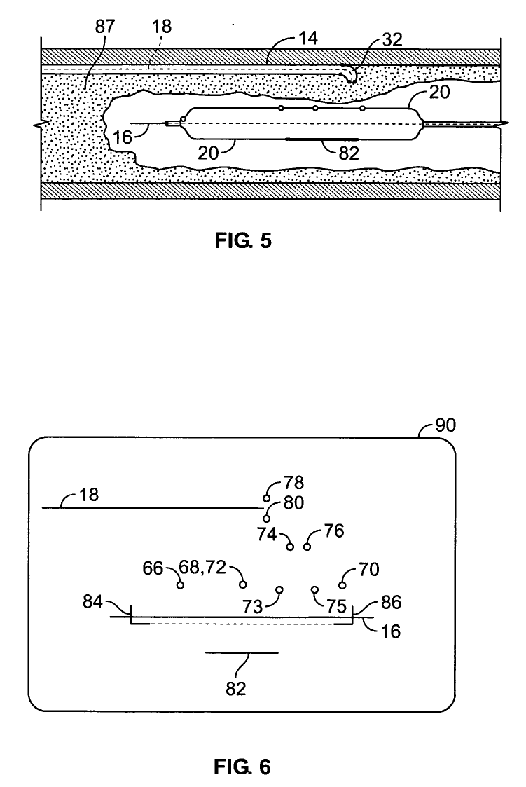

Referring to FIGS. 4 and 5, the first guide-wire 16, placed percutaneously, is advanced downstream through a vascular body or structure 86 (e.g., an arterial lumen) to a treatment site 87 (referred to hereinafter as “the CTO region”) where a CTO is present. If the CTO region 87 is present in a lower extremity of a patient, the first guide-wire 16 is preferably introduced into the vascular structure 86 through a puncture made at a patient's thigh portion. Once the first guide-wire 16 is properly positioned, the balloon 20 is then advanced along the first guide wire 16 until it is until it is positioned adjacent the CTO region 87 (see FIG. 4). A second guide-wire 18 is also introduced into the vascular structure 86 from an area distal to the CTO region 87 (e.g., from an incision made in a patient's ankle or foot portion if the CTO region 87 is in a lower extremity of the patient). The second guide-wire 18 is advanced upstream to the CTO region 87 to a point just distal thereto. A conventional straight catheter 88, used in conjunction with the second guide-wire 18, is advanced upstream through the CTO region 87, in the plane of dissection (see FIG. 4). The catheter 88 facilitates the passage of guide-wire 18 through the plane of dissection, as it crosses the CTO region 87.

As shown in FIG. 5, the straight catheter 88 has been replaced by the angled catheter 14 along the second guide-wire 18. More particularly, the straight catheter 88 is withdrawn from the CTO region 87 by being pulled along the second guide-wire and exiting through the skin of the patient at its original point of entry, leaving just the second guide-wire 18 in place within the vascular structure 86. The angled catheter 14 is then introduced to the patient via the point of entry used by the straight catheter 88, and advanced over the second guide-wire 18. The tapered end portion 32 of the angled catheter 14 is preferably made from an elastic material such that the tapered end portion 32 can be oriented from its normal, angled orientation (as shown in FIG. 2) to a substantially linear orientation relative to the elongate portion 30. As a result, the tapered end portion 32 can be passed through the CTO region 87 in its linear orientation so as to facilitate passage therethrough. Also, it is preferred that the tapered end portion 32 of the angled catheter 14 and the funnel-shaped opening 60 (FIG. 2) of the balloon 20 are nearly complementary in shape (for example, conical shape) such that the tapered end portion 32 can be “popped” into the funnel-shaped opening 60 upon inflation of the balloon 20. However, it should be understood by persons of ordinary skill in the art that the complementary shape is merely a preference, and is not required for proper operation of the invention.

After positioning the balloon 20 and the tapered end portion 32 of the angled catheter 14 at the CTO region 87, the axial and angular orientation of the balloon 20 and/or the tapered end portion 32 of the angled catheter 14 is adjusted for proper alignment/positioning. Referring to FIGS. 3 and 6, in order to properly position the balloon 20 relative to the angled catheter 14, a practitioner can use a radioscope display 90 (see FIG. 6) to remotely view the guide-wires 16 and 18, as well as the radio-opaque markers 66, 68, 70, 72, 73, 75, 82, 84, 86 (see FIGS. 1-3) of the balloon 20 and the radio-opaque markers 74, 76, 78, 80 (see FIG. 2) of the angled catheter 14. With the aid of radioscope display 90, the balloon 20 and/or the angled catheter 14 can be moved axially and/or rotated relative to each other and/or around their respective guide-wires as necessary. For instance, images of the radio-opaque markers 84, 86 of the elongate body 22 appearing on the radioscope display 90 are used for adjusting the angular orientation of the balloon 20. More particularly, the balloon 20 is rotated until the vertical portions of the “L” shaped markers 84, 86 appear at a maximum size on the radioscope display 90. Because the markers 84, 86 are arranged on a lateral surface of the elongate body 22, if the trough 52 of the balloon 20 is not in substantial angular alignment with the angled catheter 14, one or both of the markers 84, 86 may not be visible on the radioscope display 90, or their vertical portions may appear short on same. In order to adjust the angular orientation of the balloon 20, the balloon 20 is rotated until the marker 84, 86 become visible on the radioscope display 90 and/or until the respective vertical portions of the markers 84, 86 appear with their maximum lengths on the radioscope display 90. The angular orientation of the angled catheter can be adjusted in a similar manner by viewing the radio-opaque markers 74, 76 and/or the radio-opaque makers 78, 80.

One or more of images appearing on the radioscope display 90 of the radio-opaque markers 66, 68, 70, 72, 73, 74, 75, 76, 78, 80 can also be used to verify whether the trough 52 and/or the funnel-shaped opening 60 are axially aligned with the tapered end portion 32 of the angled catheter 14. For instance, if the radio-opaque markers 74, 76 of the angled catheter 14 appear on the radioscope display 90 as being located axially between the radio-opaque markers 66, 70 of the balloon 20, such positioning indicates that the tapered end portion 32 is axially aligned with the trough 52. If such alignment is not indicated by the radioscope display 90, the angled catheter 14 and/or the balloon 20 can be moved axially to achieve proper alignment.

By the end of the alignment procedure discussed above, the tapered end portion 32 of the angled catheter 14 should be pointing directly toward the funnel-shaped opening 60 (FIG. 2) of the balloon 20, and vice versa. In this manner, when the balloon 20 is inflated, the tapered end portion 32 of the angled catheter 14 can properly engage the funnel-shaped opening 60, as will be discussed in greater detail hereinbelow.

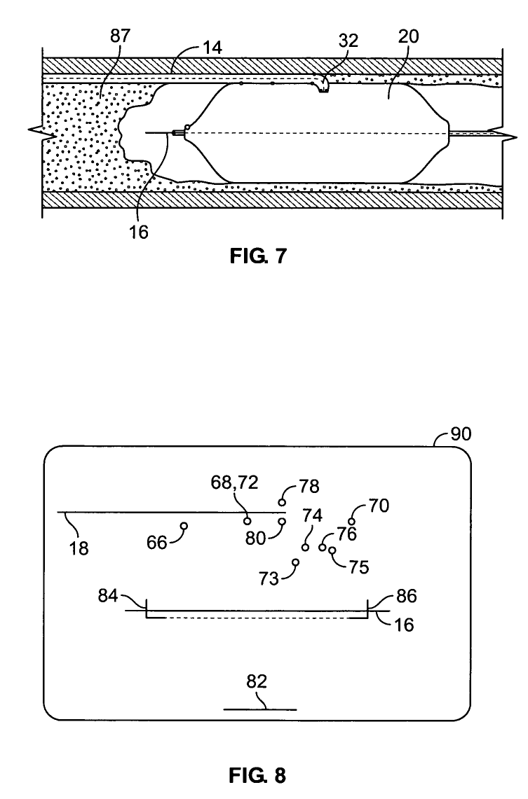

Referring now to FIG. 7, once proper alignment between the tapered end portion 32 of the angled catheter 14 and the funnel-shaped opening 60 (FIG. 2) of the balloon 20 has been achieved, the balloon 20 is inflated. Such inflation of the balloon gives form to the trough 52 (FIG. 2) of the balloon 20, and eventually causes reactive forces from the walls of the balloon 20 to force the trough 52 and the tapered end portion 32 of the catheter 14 towards each other until the latter “pops” into the funnel-shaped opening 60 of the balloon 20. To the extent a small amount of axial or angular misalignment exists between the tapered end portion 32 and the trough 52 (FIG. 2) of the balloon 20 during or after inflation of the balloon 20, the tip 34 (FIG. 2) of the tapered end portion 32 can be caused to slide longitudinally or laterally along the surface of the scalloped region 58 (FIG. 2) as necessary to mate the parts. The practitioner can use the radioscope display 90 to remotely view (see FIG. 8) the guide-wires 16 and 18, and the radio-opaque markers 66, 68, 70, 73, 74, 75, 76, 78, 80, 82, 84, 86 of the balloon 20 and the angled catheter 14, so as to confirm proper mating has occurred between the angled catheter 14 and the balloon 20.

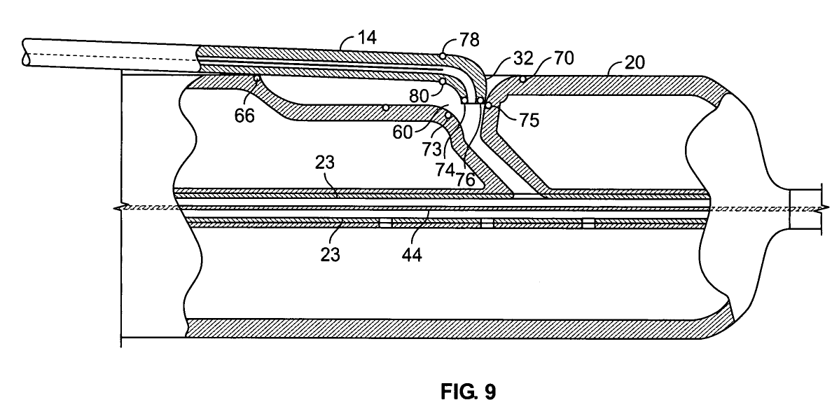

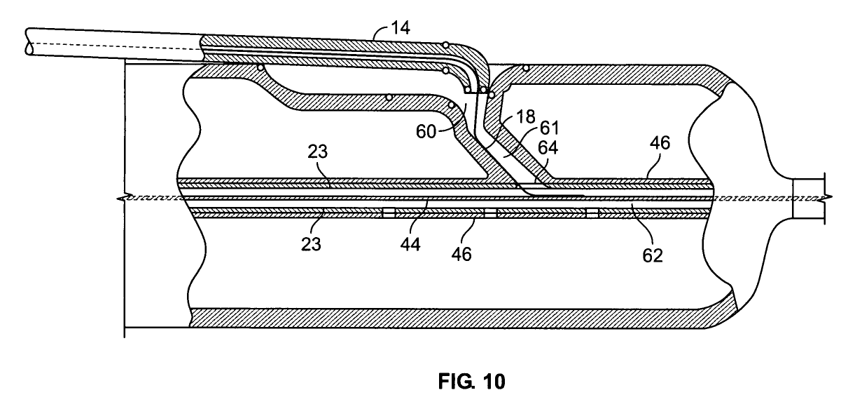

The nature of the mating relationship between the angled catheter 14 and the balloon 20 is illustrated in detail in FIG. 9. More particularly, the full and complete insertion of the tapered end portion 32 of the angled catheter 14 into the funnel-shaped opening 60 of the balloon 20, remotely confirmed by the practitioner via images appearing on the radioscope display 90, is shown in FIG. 9. (Note the similar comparative positions, as between FIGS. 6 and 8, of the guide-wires 16, 18, and the radio-opaque markers 74, 76, 78, 80, 84, 86 of the balloon 20 and the angled catheter 14.) The second guide wire 18 can now be advanced into the balloon 20, and into the lumen 23 (FIG. 2) and out of the vascular structure 86 of the patient. This process and a variation thereof will now be described below with reference to FIGS. 10-12.

As shown in FIG. 10, the first guide-wire 16 is removed from the lumen 23. This removal of the first guide-wire 16 allows for the advancement the second guide-wire 18 down the funnel-shaped opening 60 through the aperture 64 of the elongate body 22 into the lumen 23. Edges of the funnel-shaped opening 60 are adapted to permit the second guide-wire 18 to be snaked through the funnel-shaped opening 60 and into the axially disposed lumen 23 to thereby reduce the chances of second guide-wire 18 accidentally bending in a wrong direction. Due to its slanted configuration, the channel 61 facilitates the passage of the guide-wire 18 therethrough and into lumen 23.

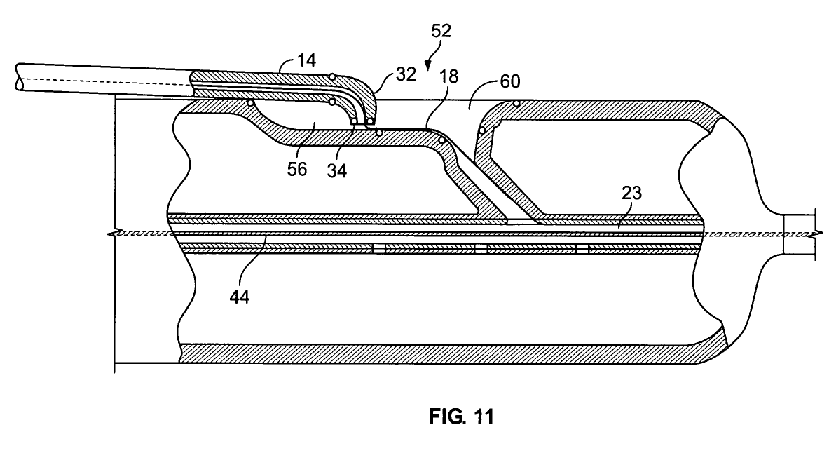

FIG. 11 illustrates a variation in the preferred alignment of the tapered end portion 32 of the angled catheter 14 with the funnel-shaped opening 60. In this particular case, the angled catheter 14 is arranged within the capture zone 56 of the trough 52, but the tapered end portion 32 of the angled catheter 14 is not in precise angular and/or axial alignment with the funnel-shaped opening 60 prior to inflation of the balloon 20, so the tapered end portion 32 has not “popped” into place in the funnel-shaped opening 60. Nevertheless, the practitioner can still advance the second guide-wire 18 into the funnel-shaped opening 60 and into the lumen 23. It is for this reason that the length of the tip 34 of the angled catheter 14 is preferably comparable to the depth of the trough 52, including but not limited to, for example, 2 mm to 2.5 mm. Also, the second guide-wire 18 should be flexible so that it can be advanced into the funnel-shaped opening 60 even though the tip 34 of the angled catheter 14 is not in its preferable position (aligned with the funnel-shaped opening 60).

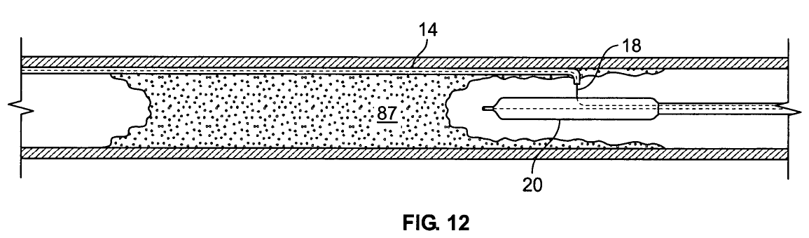

As shown in FIG. 12, the balloon 20, which has now captured the second guide-wire 18 such that the balloon 20 can now be deflated and decoupled from the angled catheter 14, is shown having returned to its uninflated state. The second guide-wire 18, a section of which is now directly visible in the space between the now-decoupled components, is advanced further upstream out of the balloon 20, through the elongate body 22 of the balloon assembly 12, and out of the vascular structure 86 of the patient such that the end 42 (see FIG. 2) of the second guide-wire 18 is outside of the patient's body and can be grasped or otherwise manipulated by the practitioner. At this point, the practitioner has a much greater ability to manipulate the axial position of the second guide-wire 18 (from two ends) than was the case when the end of the second guide-wire 18 was merely suspended in space at the upstream end of the CTO region 87 (see FIG. 4). The balloon 20 can now be removed from the vascular structure 86.

Now, although not shown, the second-guide wire 18 enters the body at a first entry point downstream of the CTO region 87 (e.g., at a foot or ankle region for treatment of a CTO in a lower extremity) and exits the body upstream of the CTO region 87 where the first guide-wire 16 entered through the skin (e.g., a thigh region for treatment of a CTO in a lower extremity). A conventional treatment balloon (not shown) can be tracked over the second guide-wire 18 from either the upstream or downstream entry points in the body (not shown). After positioning the treatment balloon in the desired location within the CTO region 87, the inflation of the treatment balloon pushes the CTO against the walls of the vascular structure 86, thus enlarging the opening made by the second guide-wire 18.

It should be noted that numerous advantages are provided by the system 10 of the present invention, and the above-described use of same to better position a treatment guide-wire relative to the axis of a vascular structure having a chronic total occlusion. For example, the number and locations of the radio-opaque markers present in the angled catheter 14 and the balloon 20 are advantageously selected and implemented so as to simplify, to the maximum extent possible, the task of the practitioner in rotating and moving the angled catheter 14 and the balloon 20 relative to each other as needed prior to coupling, and to verify proper coupling after inflation of the balloon 20. However, these markers can be rearranged, removed, or in cases, more marker can be added according to need. Also, the right-angle design embodied by the tapered end portion 44 of the angled catheter 14 and the funnel-shaped opening 60 of the balloon 20 reduces the actual coupling process to a simple “pop-in” step, according to which the practitioner need only inflate the balloon 20 toward the angled catheter 14, while simultaneously monitoring the radioscope display 90 to confirm a preferred method of coupling. Additionally, the present invention is configured to accommodate an imprecise arrangement where the tapered end portion 32 of the angled catheter 14 is positioned within the capture zone 56 of the trough 52 but not necessarily within the funnel-shaped opening 60, by allowing a practitioner to track the wire along the capture zone 56 and into the funnel-shaped opening 60. This variation in the method greatly simplifies and maximizes the chances of success in the subsequent balloon inflation/coupling step.

The system and method discussed above are particularly suitable for treating a CTO condition in a lower extremity, but the invention can be used for other vascular structures. For instance, typically, with regard to the present invention, a 4 French arterial sheath, which is known in the art (but not shown), can be placed within the lumen of the artery distal (away from the heart) to the CTO. In the lower extremity this artery is either the Posterior Tibial or Anterior Tibial Artery at the foot or ankle level. Under standard techniques the wire is advanced in a retrograde manner (going upstream) until the CTO is reached.

It will be understood that the embodiment described herein is merely exemplary and that a person skilled in the art may make many variations and modifications without departing from the spirit and scope of the invention. All such variations and modifications are intended to be included within the scope of the invention as defined in the appended claims.

What is claimed is:

1. A catheter device for insertion into a vascular body, comprising a carrier having a lumen extending therethrough; an inflatable balloon attached to said carrier so as to be carried thereby, said balloon being expandable from a deflated position to an inflated position in response to the introduction of pressurized fluid into said balloon, said balloon having an opening formed in an exterior surface 10 of said balloon, said opening permitting communication between said lumen and said exterior surface.

2. The device of claim 1, wherein said opening is sized and shaped so as to permit a guide wire to be fed from said exterior surface of said balloon into said lumen through said opening.

3. The device of claim 2, wherein said balloon includes a trough formed adjacent an outer periphery thereof when said balloon is in its said inflated position, said trough being open to said exterior surface of said balloon and communicating with said lumen through said opening.

4. The device of claim 3, wherein said opening extends in a generally radial direction and is positioned between said trough and said carrier, said carrier including a hole, said opening communicating with said lumen through said hole.

5. The device of claim 4, wherein said trough extends along a generally axial direction for receiving an end of a catheter so as to permit the introduction of a guide wire fed from the end of the catheter into said lumen of said carrier.

6. The device of claim 5, wherein said opening includes a funnel-shaped section for facilitating the introduction of a guide wire from the exterior of said balloon into said lumen, said funnel-shaped section being in direction communication with said through.

7. The device of claim 6, wherein said opening includes a channel extending from said funnel-shaped section to said hole of said carrier.

8. The device of claim 7, wherein said channel is oriented in a slanted position so as to facilitate the insertion of a guide wire from the exterior of said balloon into said lumen.

9. The device of claim 1, further comprising indicating means for indicating the orientation of said balloon within a vascular body.

10. The device of claim 9, wherein said indicating means includes first indicating means for indicating the axial orientation of said balloon within a vascular body and second indicating means for indicating the angular orientation of said balloon within a vascular body.

11. The device of claim 10, wherein said first indicating means includes a plurality of first radio-opaque markers positioned on said balloon.

12. The device of claim 11, wherein said first radio-opaque markers are positioned on said balloon around said opening.

13. The device of claim 12, wherein said second indicating means includes a plurality of second radio-opaque markers coupled to said carrier.

14. The device of claim 13, wherein at least one of said second radio-opaque markers has an L-shape.

15. The device of claim 1, wherein said lumen is sized and shaped so as to permit passage of a guide wire used for placement of said balloon at a treatment site within a vascular body.

16. The device of claim 15, wherein said lumen is divided into a plurality of passages, one of which is sized and shaped for conveying pressurized fluid to said balloon.

17. A catheter system for positioning a guide wire through a treatment site within a vascular body, comprising a first catheter having carrier, which includes a lumen extending therethrough, and an inflatable balloon, which is attached to said carrier so as to be carried thereby, said balloon being expandable from a deflated position to an inflated position in response to the introduction of pressurized fluid into said balloon, said balloon having an opening formed in an exterior surface of said balloon, said opening permitting communication between said exterior surface and said lumen; and a second catheter having a portion adjacent an end thereof, said portion being sized and shaped so as to be positioned adjacent said opening of said balloon when said balloon is in its said inflated position.

18. The system of claim 17, wherein said opening is sized and shaped such that a guide wire can be fed from said second catheter into said lumen of said first catheter through said opening.

19. The system of claim 18, wherein said balloon includes a trough formed adjacent an outer periphery thereof when said balloon is in its said inflated position, said trough sized and shaped so as to receive said portion of said second catheter, said trough communicating with said lumen through said opening.

20. The system of claim 19, wherein said opening extends in a generally radial direction and is positioned between said trough and said carrier, said carrier including a hole so as to permit communication between said opening and said lumen.

21. The system of claim 20, wherein said trough extends along a generally axial direction for receiving said portion of said second catheter so as to permit the introduction of a guide wire fed from said portion of said second catheter into said lumen of said carrier.

22. The system of claim 21, wherein said opening includes a funnel-shaped section for facilitating the introduction of a guide wire fed from said portion of said second catheter into said lumen of said first catheter, said funnel-shaped section being in direction communication with said through.

23. The system of claim 22, wherein said opening includes a channel extending from said funnel-shaped section to said hole of said carrier.

24. The system of claim 23, wherein said channel is oriented in a slated position so as to facilitate the insertion of a guide wire from said portion of said second catheter into said lumen.

25. The system of claim 17, wherein said portion defines an end portion of said second catheter.

26. The system of claim 25, wherein said end portion of said catheter includes a tip sized and shaped for alignment with at least a portion of said opening of said first catheter.

27. The system of claim 26, wherein said tip of said second catheter is oriented at an angle relative to a longitudinal axis of said second catheter.

28. The system of claim 27, wherein said tip of said second catheter is oriented at an approximately 90 degree angle relative to said longitudinal axis of said second catheter.

29. The system of claim 28, wherein said second catheter includes a lumen extending through said second catheter and terminating at said tip for allowing a guide wire to extend through said second catheter.

30. The system of claim 27, wherein said balloon includes a trough formed adjacent an outer periphery thereof when said balloon is in its said inflated position, said trough being sized and shaped so as to receive said tip of said second catheter, said trough communicating with said lumen through said opening.

31. The system of claim 17, further comprising indicating means for indicating the orientation of said balloon relative to said second catheter within a vascular body.

32. The system of claim 31, wherein said indicating means includes a plurality of first radio-opaque markers positioned on said balloon.

33. The system of claim 32, wherein said first radio-opaque markers are positioned on said balloon around said opening.

34. The system of claim 33, wherein said indicating means includes a plurality of second radio-opaque markers coupled to said carrier.

35. The system of claim 34, wherein at least one of said second radio-opaque markers has an L-shape.

36. The system of claim 35, wherein said indicating means includes a plurality of third radio-opaque markers coupled to said second catheter.

37. The system of claim 31, wherein said indicating means includes first indicating means for indicating the axial orientation of said balloon relative to said second catheter within a vascular body and second indicating means for indicating the angular orientation of said balloon relative to said second catheter within a vascular body.

38. A method for positioning a catheter guide wire through a treatment site in a vascular body, comprising the steps of:
(a) advancing a first catheter to the treatment site through the vascular body from an upstream side of the treatment site;
(b) advancing a second catheter to the treatment site through the vascular body from a downstream side of the treatment site;
(c) engaging the first catheter with the second catheter within the vascular body within the vascular body adjacent the treatment site;
(d) feeding a guide wire from one of the first and second catheters to the other one of the first and second catheters; and
(e) removing the first and second catheters from the vascular body, thereby leaving the guide wire extending through the treatment site.

39. The method of claim 38, wherein the first catheter includes an inflatable balloon and wherein step (c) includes the step of inflating the balloon such that the second catheter is engaged with the balloon.

40. The method of claim 39, wherein step (c) is performed by feeding the guide wire from said second catheter to said first catheter.
